(19) United States
(12) Patent Application Publication
(10) Pub. No.: US 2020/0172843 A1
(43) Pub. Date: Jun. 4, 2020

(54) APPARATUS FOR CONTROLLING AN EXPERIMENTAL TEMPERATURE OF AN EXPERIMENTAL MATERIAL

(71) Applicant: Arctoris Limited, Oxford (GB)

(72) Inventors: Alice Poppy Roworth, Oxford (GB); Martin-Immanuel Bittner, Romsey (GB)

(21) Appl. No.: 16/209,467

(22) Filed: Dec. 4, 2018

(51) Int. Cl. C12M 1/38; C12M 1/00; C12M 3/00; C12N 5/00; C12M 1/36; B01L 7/00; B01L 7/04

(52) U.S. Cl. CPC C12M 1/38; C12M 1/005; C12M 3/00; C12N 5/0018; B01L 2300/0609; C12M 41/48; B01L 7/52; B01L 7/04; B01L 2300/1883; C12M 41/14

(57) ABSTRACT

An apparatus for controlling an experimental temperature of experimental material is disclosed. The apparatus can include a structural holder structured to hold the experimental material, the structural holder including a surface to receive the experimental material. The apparatus can also include a first layer, a second layer and a third layer of respectively a phase change material, a reflective material and an insulating material, the first layer disposed on the surface of the structural holder, the second layer disposed on the first layer, and the third layer disposed on the second layer, at least the first layer of the phase change material being configured to control the experimental temperature of the experimental material when held by the structural holder.

### TECHNOLOGICAL FIELD

The present disclosure relates generally to cell culturing and, in particular, to an apparatus for controlling an experimental temperature of an experimental material, such as cell cultures.

### BACKGROUND

Cell culture is the process by which cells are grown under controlled conditions, generally outside their natural environment. After the cells of interest have been isolated from living tissue, they can subsequently be maintained or frozen for long term storage under carefully controlled conditions in incubators (e.g., temperatures of about 37 degrees Celsius), which may vary for each cell type. When working with the cells of interest, it is desirable to maintain the controlled conditions of the cells in order to prevent damage or changes in normal cellular processes thereto. However, it may be difficult to maintain the controlled conditions of the cells when manipulation of the cells is desired. For example, when working with mammalian cells, either from cultured cell lines, or primary cells isolated from individuals, it may be desirable to maintain these cells at body temperature (e.g., about 37° C.). Even short exposure to temperatures slightly below body temperature can result in less than desirable experimental results. In this example, even at mild hypothermic conditions (e.g., about 32° C.), mammalian cells may go into “cold shock”, which may permanently affect the mammalian cells even after the mammalian cells return to body temperature.

Another controlled condition that may be difficult to maintain may include preventing evaporation and condensation from forming in experimental receptacles, such as microplates. For example, as the experimental receptacle holding the cell culture cools, condensation from growth media, in which the cell culture is supported, may form within the experimental receptacle. This has the effect of lowering a volume of growth media, which therefore alters a concentration of any additive that has been added (for example drugs). As getting a correct concentration of a drug in the experimental receptacle is desirable for ensuring an effectiveness of the experiment, ensuring reproducibility between experiments, and making informed decisions about downstream applications, any change in condensation in the experimental receptacle will impact these factors. Condensation formation in the experimental receptacle (e.g., on a lid of a microplate) can also inhibit microscopy as the droplets impede visuals.

Still another controlled condition that may be difficult to maintain may include ambient temperatures in each laboratory setting. For example, each laboratory may have a different ambient temperature, which may also be variable within the same laboratory depending on a time of year, a time of day, a presence of windows, an effectiveness of air conditioning, a position of the air conditioning (e.g., the workbench being directly under the air conditioning vent may be cooler compared to a workbench at the other end of the room), and the like. Therefore, ambient temperature is not a standardized temperature that is maintainable across each experiment.

Scientists are attempting to maintain controlled conditions by minimizing time that cell cultures spend out of incubators. However, since any time spent out of incubators has a potential to subject the cell cultures to damage due to time spent away from the controlled conditions; such exposure, even short exposure, can result in less than desirable experimental results. This, impacts the reproducibility of experiments.

Accordingly, it may be desirable to have an apparatus for controlling an experimental temperature of an experimental material, such as cell cultures, which addresses the issues noted herein.

### BRIEF SUMMARY

Example implementations of the present disclosure are directed to an apparatus for controlling an experimental temperature of experimental material where the experimental temperature is adjusted to meet a wide variety of target experimental temperatures, as required by the experiment, to establish the new target experimental temperature, and to maintain the target experimental temperature, without damaging the experimental material.

The present disclosure thus includes, without limitation, the following example implementations.

Some example implementations provide an apparatus for controlling an experimental temperature of experimental material, the apparatus comprising a structural holder structured to hold the experimental material, the structural holder including a surface to receive the experimental material; and a first layer, a second layer and a third layer of respectively a phase change material, a reflective material and an insulating material, the first layer disposed on the surface of the structural holder, the second layer disposed on the first layer, and the third layer disposed on the second layer, at least the first layer of the phase change material being configured to control the experimental temperature of the experimental material when held by the structural holder.

In some example implementations of the apparatus of any preceding example implementation, or any combination of any preceding example implementations, the phase change material comprises an indicator configured to produce a visible indication when a temperature of the phase change material deviates from a target experimental temperature.

In some example implementations of the apparatus of any preceding example implementation, or any combination of any preceding example implementations, the indicator comprises a plurality of microcapsule objects dispersed throughout the phase change material, and containing a thermochromatic dye configured to change color as the temperature of the phase change material deviates from the target experimental temperature.

In some example implementations of the apparatus of any preceding example implementation, or any combination of any preceding example implementations, the microcapsule objects have a density lower than the phase change material and thereby float to a surface of the phase change material as the temperature of the phase change material deviates from the target experimental temperature.

In some example implementations of the apparatus of any preceding example implementation, or any combination of any preceding example implementations, the structural holder comprises a base including the surface and a lid configured to engage the base, the base being structured to receive the experimental material on the surface.

In some example implementations of the apparatus of any preceding example implementation, or any combination of any preceding example implementations, either or both the base or the lid includes the first layer, the second layer and the third layer.

In some example implementations of the apparatus of any preceding example implementation, or any combination of any preceding example implementations, one or more of the first layer, the second layer or the third layer is contained within a sterilizable material.

In some example implementations of the apparatus of any preceding example implementation, or any combination of any preceding example implementations, the structural holder further includes a raised edge extending about the surface thereof, the first layer being disposed on the surface and the raised edge.

In some example implementations of the apparatus of any preceding example implementation, or any combination of any preceding example implementations, the apparatus further comprises a microplate defining a plurality of wells configured to receive the experimental material therein, the microplate being received on the surface of the structural holder, in contact with the first layer on the surface and the raised edge.

In some example implementations of the apparatus of any preceding example implementation, or any combination of any preceding example implementations, the microplate is integrally formed with the structural holder such that the first layer is in thermal conductive contact with a bottom surface of the microplate including the plurality of wells.

In some example implementations of the apparatus of any preceding example implementation, or any combination of any preceding example implementations, the apparatus further comprises a flask configured to receive the experimental material therein, and having a height equal the raised edge of the structural holder, the flask being received on the surface of the structural holder within the raised edge thereof, in contact with the first layer on the surface and the raised edge.

In some example implementations of the apparatus of any preceding example implementation, or any combination of any preceding example implementations, the apparatus further comprises a bottle configured to receive the experimental material therein, and having a height equal the raised edge of the structural holder, the bottle being received on the surface of the structural holder within the raised edge thereof, an in contact with the first layer on the surface and the raised edge.

In some example implementations of the apparatus of any preceding example implementation, or any combination of any preceding example implementations, the apparatus further comprises a mold for centrifuge vials or microtubes configured to receive the experimental material therein, and having a height equal the raised edge of the structural holder, the mold being received on the surface of the structural holder within the raised edge thereof, in contact with the first layer on the surface and the raised edge.

In some example implementations of the apparatus of any preceding example implementation, or any combination of any preceding example implementations, the phase change material is selected from the group consisting of at least a hydrated salt phase change material, an organic phase change material, and a eutectic phase change material.

In some example implementations of the apparatus of any preceding example implementation, or any combination of any preceding example implementations, the reflective material is selected from the group consisting of at least metallized polyethylene terephthalate (MPET).

In some example implementations of the apparatus of any preceding example implementation, or any combination of any preceding example implementations, the insulating material is selected from the group consisting of at least vacuum insulated panels (VIPs), expanded polystyrene (EPS) foam, and polyurethane (PUR) foam.

In some example implementations of the apparatus of any preceding example implementation, or any combination of any preceding example implementations, the second layer of the reflective material comprises two sheets of reflective material defining an air gap therebetween from at least one of a honeycomb core provided between the two sheets of reflective material and a quantity of pressurized air directed between the two sheets of reflective material.

In some example implementations of the apparatus of any preceding example implementation, or any combination of any preceding example implementations, the phase change material is configured to control the experimental temperature of the experimental material based on at least a current and a target experimental temperature of the experimental material, an initial temperature, a melting point temperature of the phase change material, a mass of the phase change material relative to a mass of the experimental material, thermal properties of the phase change material, and thermal conduction between the phase change material and the experimental material through the structural holder configured to hold the experimental material.

In some example implementations of the apparatus of any preceding example implementation, or any combination of any preceding example implementations, the melting point temperature of the phase change material is the target experimental temperature of the experimental material so that the experimental temperature of the experimental material is maintained at the melting point temperature when the experimental material is held by the structural holder.

In some example implementations of the apparatus of any preceding example implementation, or any combination of any preceding example implementations, the melting point temperature of the phase change material is the target experimental temperature of the experimental material, and the temperature and the melting point temperature of the phase change material, are adjustable in order to adjust the experimental temperature of the experimental material when the experimental material is held by the structural holder.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURE(S)

Having thus described the disclosure in general terms, reference will now be made to the accompanying figures, which are not necessarily drawn to scale, and wherein:

FIGS. 1A and 1B illustrate different views of a schematic of an example apparatus for controlling an experimental temperature of an experimental material according to example implementations of the present disclosure;

FIG. 2 illustrates a detail view of an example first layer of a phase change material with an indicator according to example implementations of the present disclosure;

FIG. 3 illustrates a detail view of an example second layer of a reflective material according to example implementations of the present disclosure;

FIGS. 4A-4C illustrate different views of an example apparatus for controlling an experimental temperature of an experimental material, the experimental material being received in a microplate according to example implementations of the present disclosure;

FIG. 5 illustrates an example apparatus for controlling an experimental temperature of an experimental material, the experimental material being received in a microplate integral with the apparatus according to example implementations of the present disclosure;

FIGS. 6A and 6B illustrate different views of an example apparatus for controlling an experimental temperature of an experimental material, the experimental material being received in a flask according to example implementations of the present disclosure;

FIG. 7 illustrates an example apparatus for controlling an experimental temperature of an experimental material, the experimental material being received in a bottle according to example implementations of the present disclosure; and

FIGS. 8A-8C illustrate different views of an example apparatus for controlling an experimental temperature of an experimental material, the experimental material being received in a mold for centrifuge vials or microtubes according to example implementations of the present disclosure.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be expressed in many different forms and should not be construed as limited to the implementations set forth herein; rather, these example implementations are provided so that this disclosure will be thorough and complete, and will fully convey the scope of the disclosure to those skilled in the art. As used herein, the term “and/or” and the “/” symbol includes any and all combinations of one or more of the associated listed items. Also, for example, reference may be made herein to quantitative measures, values, relationships or the like. Unless otherwise stated, any one or more if not all of these may be absolute or approximate to account for acceptable variations that may occur, such as those due to engineering tolerances or the like.

Further, unless otherwise indicated, something being described as being a first, second or the like should not be construed to imply a particular order. It should be understood that the terms first, second, etc. may be used herein to describe various steps, calculations, positions and/or the like, these steps, calculations or positions should not be limited to these terms. These terms are only used to distinguish one operation, calculation, or position from another. For example, a first position may be termed a second position, and, similarly, a second step may be termed a first step, without departing from the scope of this disclosure. Additionally, something may be described as being above something else (unless otherwise indicated) may instead be below, and vice versa; and similarly, something described as being to the left of something else may instead be to the right, and vice versa. As used in the specification, and in the appended claims, the singular forms “a,” “an,” “the,” include plural referents unless the context clearly dictates otherwise. Like reference numerals refer to like elements throughout.

Example implementations of the present disclosure provide an apparatus for controlling an experimental temperature of an experimental material. As used herein, an “experimental material” may comprise mammalian cells, either from cultured cell lines, or primary cells isolated from individuals, non-mammalian animal cells, plant cells, fungal cultures, microbiological cultures of microbes, chemical mixtures where temperature affects rate and outcome of chemical reactions, etc. Any other type of cell that may be grown under controlled conditions, outside its natural environment, can also be considered “experimental material” as used in this disclosure.

In some example implementations, the apparatus controls an experimental temperature of an experimental material. The “experimental temperature” of the experimental material may be a temperature of the experimental material, which may be maintained, adjusted, established or otherwise controlled by the apparatus from an initial temperature of the experimental material. In some examples, a target experimental temperature or a desired experimental temperature may be a temperature set point in the experiment which the experimental temperature of the experimental material is controlled to either be maintained at and/or adjusted to reach by increasing or decreasing the experimental temperature of the experimental material from the initial temperature of the experimental material.

In some example implementations, the target experimental temperature in an experiment may be a body temperature or initial temperature of the experimental material. For mammalian cells, the body temperature may be about 37 degrees Celsius (° C.), for insect cells the body temperature may be about 27° C., for avian cells the body temperature may be about 38.5° C., for amphibian cells the body temperature may be between about 15 and about 26° C., etc. In other examples, the initial temperature of the experimental material may be a temperature of a storage facility of the experimental material, such as a freezer (e.g., about −21° C.), while the target experimental temperature is significantly higher (e.g., about 21° C. or ambient temperature). In still other examples, the initial temperature of the experimental material may be the body temperature of the experimental material, while the target experimental temperature is lower (e.g., a temperature of a room, such as an ambient temperature, e.g., about 21° C.). Therefore, the apparatus as disclosed herein may be advantageously configured to control the experimental temperature of the experimental material so that the experimental material is able to be adjusted from the initial temperature to meet a wide variety of target experimental temperatures, as required by the experiment, to establish the new target experimental temperature, and to maintain the target experimental temperature, without damaging the experimental material.

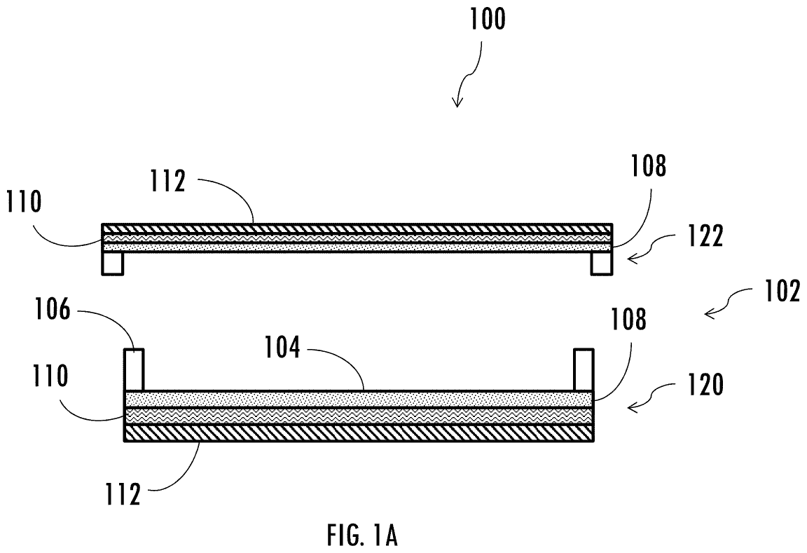

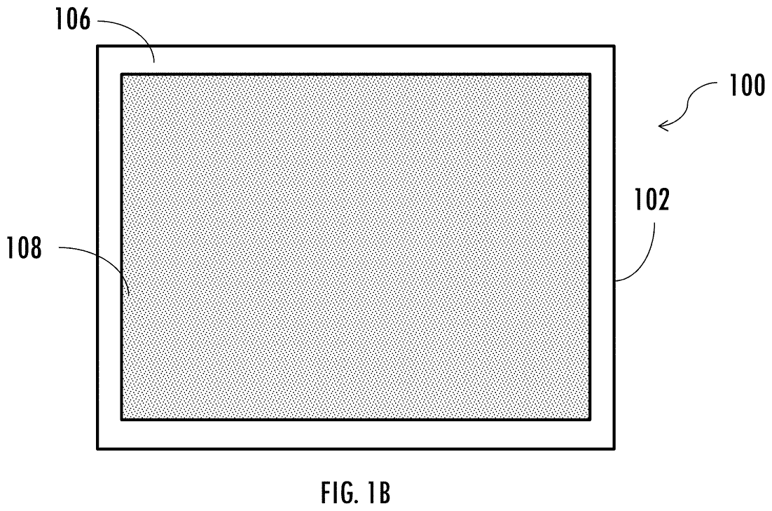

FIGS. 1A and 1B illustrate different views of an example apparatus for controlling an experimental temperature of an experimental material, generally referred to as 100, according to example implementations of the present disclosure. The apparatus may be used to control the experimental temperature of any type of experimental material as disclosed herein, including but not limited to mammalian cells, non-mammalian animal cells, plant cells, fungal cultures, microbiological cultures of microbes, chemical mixtures where temperature affects rate and outcome of chemical reactions, etc. As shown, the apparatus comprises a structural holder 102 structured to hold the experimental material. The structural holder includes a surface 104 configured to receive the experimental material.

More particularly, the structural holder 102 including the surface 104 is able to support the experimental material and/or any experimental receptacle configured to receive the experimental material therein. For example, the experimental receptacle may comprise a microplate, a flask, a reagent bottle, a mold for centrifuge vials or microtubes, and the like. In another example, the experimental receptacle is integrally formed with the structural holder (e.g., a microplate is integrally formed with the structural holder). In some example implementations, the experimental receptacle retaining the experimental material may be received on the surface of the structural holder so that the experimental material is arranged to be in contact with the surface of the structural holder. For example, the experimental material may be in direct or indirect physical contact with the surface of the structural holder, depending on the configuration of the experimental receptacle.

In some example implementations, the structural holder 102 may further include a raised edge 106 extending about the surface 104. The raised edge may extend about a perimeter of the surface. As illustrated in FIGS. 1A and 1B, for example, the raised edge extends substantially perpendicular relative to the surface of the structural holder. However, the raised edge may extend at an angle towards or away from a centrally defined axis of the structural holder. Further, in some example implementations, the raised edge may have a height equal or substantially equal a height of the experimental receptacle received on the surface of the structural holder.

The apparatus 100 further comprises a first layer 108, a second layer 110 and a third layer 112 of respectively a phase change material, a reflective material and an insulating material. In some example implementations, the first layer is disposed on the surface 104 of the structural holder 102, the second layer is disposed on the first layer, and the third layer is disposed on the second layer. In this manner, the first layer, the second layer, and the third layer may be arranged to facilitate heat transfer between the phase change material of the first layer and the experimental material through conductive contact while minimizing heat transfer between the phase change material and the surrounding environment through radiation or conduction.

The phase change material of the first layer 108 may be a temperature responsive material having the ability to exhibit physiochemical or mechanical changes in the presence of small temperature differences. Typically, a substance (such as water) may exhibit a phase transition at certain pre-determined temperatures. The phase transition may comprise a change between a liquid and a solid or a solid and a gas. The certain pre-determined temperatures may include a melting point temperature and/or a freezing point temperature. As used herein, the term “melting point temperature” should be construed to generally refer to the melting point temperature for a phase transition from solid to liquid, and the “freezing point temperature” for a phase transition from liquid to solid. Although melting point temperatures and freezing point temperatures are most often the same temperature, they may, in rare circumstances, differ for some substances. Nevertheless, as used herein a “phase transition temperature,” “melting point temperature,” and “freezing point temperature,” generally refer to the same temperature such that these terms may be used interchangeably or, at times, may simply be referred to as the “melting point temperature.”

Phase change materials are commonly divided into three categories, which may include, but are not limited to hydrated salt phase change materials, organic phase change materials, and eutectic phase change materials. A hydrated salt phase change material may comprise a fluid, such as water, and a salt where the choice of salt and a relative percentage of salt and water by total weight percent of the solution determines the melting/freezing point temperature thereof. An organic phase change material may comprise a paraffin and/or an oil, where the molecular composition of the organic phase change material determines the melting/freezing point temperature thereof.

A eutectic phase change material may comprise a mixture of two or more components, which do not usually interact to form a new chemical compound but, which at certain ratios, inhibit the crystallization process of one another resulting in a system having a lower melting point temperature than either of the two or more components. In some example implementations, a eutectic phase change material may be a temperature-responsive polymer that is in a liquid state when refrigerated and a solid state when at room temperature. Examples of thermogel materials may include, but are not limited to, poly (N-isopropylacrylamide), poly (N, N-diethylacrylamide), poly(methyl vinyl ether), and poly(N-vinylcaprolactam). Additional examples of temperature-responsive polymers may include, but are not limited to, PEO-b-PPO having lower critical solution temperature (LCST) values of 32° C., 33° C., 37° C., 33° C. and about 29-85° C., and PAAm/PAAc IPN having upper critical solution temperature (UCST) values of 25° C.

Commercial phase change materials, e.g., hydrated salt phase change materials, organic phase change materials, and eutectic phase change materials, are available over a standard range of melting/freezing point temperatures, for example, 0° C., 4° C., 21° C. (ambient temperature), 37° C. (body temperature for mammals), and the like. As such, in some example implementations, a commercial phase change material may be utilized where the target experimental temperature is the same as one of the standard range of melting/freezing point temperatures for phase change materials. However, it may be desirable, in some other example implementations, to utilize a phase change material with a non-standard melting/freezing point temperature where the target experimental temperature is not one of the standard range of melting/freezing point temperatures for phase change materials.

For example, where the target experimental temperature is −21° C., then a phase change material with a non-standard melting/freezing point temperature of −21° C. may be required. In order to obtain a phase change material with such a non-standard melting/freezing point temperature, in some example implementations, one or more factors or characteristics of the phase change material and/or the experimental material may be taken into consideration. The one or more factors or characteristics of the phase change material and/or the experimental material may comprise, but are not limited to, a target experimental temperature of the experimental material, a melting/freezing point temperature of the phase change material, a mass of the phase change material relative to a mass of the experimental material, and a percent by weight of components of the phase change material.

In such circumstances, the relative percentage of the constituent components of the phase change material may be adjusted to obtain the desired non-standard melting/freezing point temperature. For example, where the phase change material is a hydrated salt phase change material, the percent by weight of the salt component in the hydrated salt phase change material may be adjusted to obtain the desired non-standard melting/freezing point temperature. Otherwise, a custom phase change material may be prepared, with the desired non-standard melting/freezing point temperature.

Accordingly, the phase transition of the phase change material may comprise a transition from one phase to another (e.g., from a solid to a liquid and vice versa), the temperature at which the phase transition occurs being dependent on the defined melting/freezing point temperature. As noted herein, in some example implementations, the phase change material may define a melting point temperature that is a same as or substantially a same as the target experimental temperature, whether or not the target experimental material is one of the standard range of melting/freezing point temperatures for phase change materials. For example, if the target experimental temperature is a body temperature of mammalian cells or about 37° C., the thermogel material may define a melting point temperature of 37° C.

As such, by defining the melting/freezing point temperature of the phase change material to be the same as the target experimental temperature, the phase change material may be configured to control the experimental temperature of the experimental material to reach and maintain the target experimental temperature. The phase change material may be configured to control the experimental temperature of the experimental material by selecting or configuring the phase change material to have a melting/freezing point temperature the same as or substantially the same as the target experimental temperature and thereby cool the experimental material to the target experimental temperature (initial temperature of the experimental material is higher than the target experimental temperature), heat the experimental material to the experimental temperature (initial temperature of the experimental material is lower than the target experimental temperature), or maintain the experimental material at the experimental temperature (initial temperature and target experimental temperature are the same).

In some example implementations, the phase change material is configured to control (e.g., adjust and/or maintain) the experimental temperature of the experimental material based on at least a current and a target experimental temperature of the experimental material, an initial temperature of the phase change material, a temperature and a melting point temperature of the phase change material, a mass of the phase change material relative to a mass of the experimental material, thermal properties of the phase change material (e.g., specific heat and heat of fusion), and thermal conduction between the phase change material and the experimental material through the structural holder configured to hold the experimental material.

Accordingly, heat exchange between the phase change material and the experimental material may be determined by at least the current temperature of the experimental material, the temperature and melting point temperature of the phase change material, the mass of the phase change material relative to the mass of the experimental material, thermal properties of the phase change material, and thermal conduction between the phase change material and the experimental material through the structural holder, so as to control the temperature of the experimental material.

More particularly, as the temperature of the phase change material nears and then reaches the target experimental temperature (which may be the defined melting/freezing point temperature), the phase change material may begin a phase transition. Nearing the melting/freezing point temperature, the phase change material may begin to solidify or liquefy and, upon reaching the target experimental temperature, may remain at the target experimental temperature (which is the defined melting/freezing point temperature of the phase change material) until the last molecule of the phase change material has solidified or liquefied, respectively. After the last molecule of the phase change material changes phase, the phase change material may then continue increasing or decreasing in temperature. The different temperatures of the phase change material corresponding to the different phase transitions thereof, will likewise increase or decrease the experimental temperature of the experimental material through heat transfer (e.g., conduction or radiation).

For example, in some example implementations, the current experimental temperature of a given experimental material can be maintained at a given target experimental temperature by heat exchange (e.g. conductive contact) with a phase change material of the first layer 108 selected to have a melting/freezing point temperature the same as the target experimental temperature. The initial temperature of the phase change material may be substantially equal to the target experimental temperature and the mass of the phase change material relative to the mass of the experimental material may be determined by the thermal properties of the phase change material (e.g., specific heat and heat of fusion), the current (i.e., target) experimental temperature of the experimental material, the thermal conduction between the phase change material and the experimental material through the structural holder, and heat lost or gained by the experimental material over time by other means (e.g., conduction, convection, evaporation, condensation, etc.)

In this example, the phase change material may be configured to solidify or liquefy at the melting/freezing point temperature so that the experimental temperature of the experimental material is maintained at the melting/freezing point temperature when the experimental material is held by the structural holder 102. This may be advantageous where the experimental material is held in, for example, an incubator, a freezer, etc., at a target experimental temperature and it is desirable to maintain the experimental temperature of the experimental material at the target experimental temperature throughout the experiment when the experimental material is removed from the controlled environment of, for example, a freezer, an incubator, etc.

In another example, in some example implementations, the current experimental temperature of a given experimental material can be lowered to a given target experimental temperature by heat exchange (e.g., conductive contact) with a phase change material of the first layer 108 selected to have a melting/freezing point temperature the same as the target experimental temperature. The initial temperature of the phase change material may be at or below the target temperature, and the initial temperature of the phase change material and the mass of the phase change material relative to the mass of the experimental material may together be determined by at least the thermal properties of the phase change material (e.g., specific heat and heat of fusion), the current experimental temperature of the experimental material, and the thermal conduction between the phase change material and the experimental material through the structural holder.

In this example, a phase change material that is in a solid state at a temperature lower than a defined melting/freezing point temperature and a liquid state at a temperature higher than the defined melting/freezing point temperature may be used to cool an experimental material having an initial temperature that is higher than a target experimental temperature; the phase change material having an initial temperature that is lower than the defined melting/freezing point temperature (i.e., is in the solid state) and a melting/freezing point temperature defined to be the target experimental temperature. In this example, the phase change material in the solid state may be used to cool the experimental material from the initial temperature to the target experimental temperature and maintain the experimental material at the target experimental temperature until the phase change material has warmed to a temperature higher than the melting/freezing point temperature and thus changed from a solid to a liquid.

In a still further example, in some example implementations, the current experimental temperature of a given experimental material can be raised to a given target experimental temperature by heat exchange (e.g., conductive contact) with a phase change material of the first layer 108 selected to have a melting/freezing point temperature the same as the target experimental temperature. The initial temperature of the phase change material may be at or above the target experimental temperature and the initial temperature of the phase change material and the mass of the phase change material relative to the mass of the experimental material may together be determined by at least the thermal properties of the phase change material (e.g., specific heat and heat of fusion), the current experimental temperature of the experimental material, and the thermal conduction between the phase change material and the experimental material through the structural holder.

In this example, the phase change material that is in a solid state at a temperature lower than a defined melting/freezing point temperature and a liquid state at a temperature higher than the defined melting/freezing point temperature may be used to heat an experimental material having an initial temperature that is lower than a target experimental temperature; the phase change material having an initial temperature that is higher than the defined melting/freezing point temperature (i.e., is in the liquid state) and a melting/freezing point temperature defined to be the target experimental temperature. In this example, the phase change material in the liquid state may be used to heat the experimental material from the initial temperature to the target experimental temperature and maintain the experimental material at the target experimental temperature until the phase change material has cooled to a temperature lower than the melting/freezing point temperature and thus changed from a liquid to a solid.

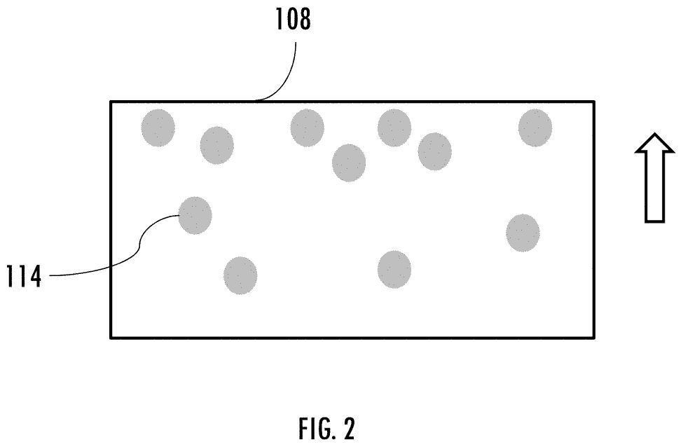

In some example implementations, the phase change material may comprise an indicator configured to produce an indication when a temperature of the phase change material deviates from the target experimental temperature, e.g., is below (lower) or above (higher) than the target experimental temperature. For example, as illustrated in FIG. 2, the indicator may comprise a plurality of microcapsule objects 114 dispersed throughout the phase change material. The microcapsule objects may comprise an outer shell containing a thermochromatic dye or a substance that incrementally changes color due to a change in temperature. The microcapsule objects may thus be configured to change color as the temperature of the phase change material deviates from the target experimental temperature, by, for example, decreasing below or increasing above the target experimental temperature.

For example, the thermochromatic dye may be configured to change color in defined increments (e.g., 4° C.) increments from the target experimental temperature. In this example, if the target experimental temperature is 37° C., such that the melting/freezing point temperature of the phase change material is also 37° C., the microcapsules containing the thermochromatic dye may change color as soon as the thermochromatic dye deviates from the target experimental temperature of 37° C., e.g., cools to 33° C. or heats to 41° C.

In addition, the microcapsule objects may comprise a density lower than the phase change material so that the microcapsule objects may thereby float to a surface of or be otherwise suspended within the phase change material. The microcapsule objects may be selected based on the density of the microcapsule objects compared to the density of the phase change material.

For example, where the target experimental temperature is 37° C., such that the melting/freezing point temperature of the phase change material is also 37° C., then at temperatures higher or lower than the melting/freezing point temperature, the phase change material may begin to change phase. Where the change in phase is from a liquid to a solid, the microcapsule objects may be able to easily move through the phase change material to float to the surface thereof, such that when the phase change material is a solid, the microcapsule objects are suspended within the solidified phase change material towards a surface thereof. The appearance of the colored microcapsule objects at the surface of the phase change material or suspended therein may provide a visible indication or perceptible effect of the change in temperature from the target experimental temperature to human scientists and/or electronic imaging system users. As such, the experimental material received in the structural holder 102 may be returned to the incubator, prior to the experimental temperature of the experimental material decreasing to the point where damage may be incurred to the experimental material.

Example thermochromatic dyes that may be used in the plurality of microcapsule objects may include, but are not limited to, lueco dyes such as spirolactones, fluorans, spiropyrans, and fulgides mixed with crystal violet lactone, weak acids, and a dissociable salt dissolved in dodecanol. Other thermochromatic dyes may include, but are not limited to, liquid crystals, inorganic compounds (e.g., cuprous mercury iodide (Cu₂[HgI₄])), and polymers (e.g., thermoplastics).

Returning back to FIGS. 1A and 1B, the reflective material of the second layer 110 may work in conjunction with the phase change material of the first layer 108 to control the experimental temperature of the experimental material. The reflective material may be a material that is capable of reflecting heat radiated from the experimental material or the phase change material back to the phase change material or the experimental material. In some example implementations, the reflective material may comprise a heat resistant sheet or film material with a metallic infra-red reflecting agent coated thereon. For example, the reflective material may comprise a plastic such as metallized polyethylene terephthalate (MPET), where the heat resistant sheet or film material may comprise a polymer (e.g., polyethylene terephthalate (PET)) and the metallic reflecting agent may comprise aluminum, so that about 97% of radiated heat is reflected back to the experimental material or the phase change material. However, other reflective materials that are capable of reflecting substantially the same percentage of irradiated heat as MPET are also contemplated herein.

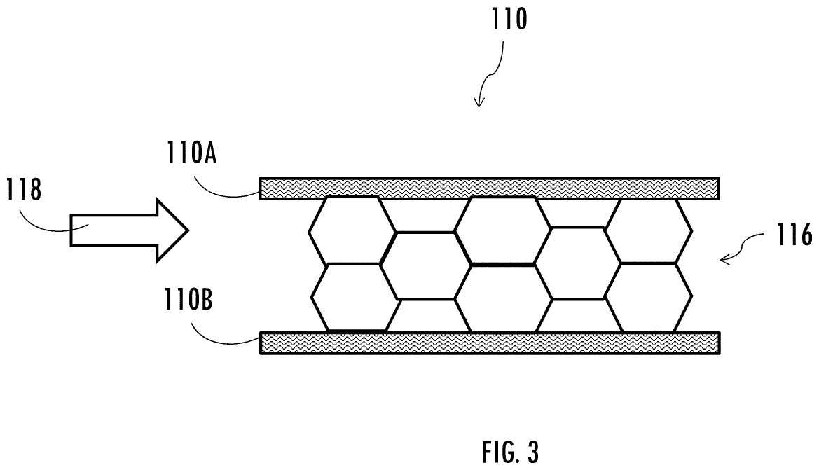

In some example implementations, and as illustrated in FIG. 3, the second layer 110 may comprise two sheets of reflective material 110A, B defining an air gap therebetween in order to reduce direct thermal conductivity. The air gap defined between the two sheets of reflective material may be formed from at least one of a honeycomb core 116 provided between the two sheets of reflective material and a quantity of pressurized air 118 directed between the two sheets of reflective material. The honeycomb core may be arranged so that the cells of the core are parallel to the reflective material or orthogonal to the reflective material. As illustrated in FIG. 3, the honeycomb core is arranged orthogonal to the reflective material. In some example implementations, both the honeycomb core and the pressurized air are utilized, but only one of the honeycomb core and the pressurized air may be utilized to provide the air gap between the two sheets of reflective material. Where both the pressurized air and the honeycomb core are utilized, the pressurized air may be directed into individual cells of the honeycomb core.

Returning back to FIGS. 1A and 1B, the insulating material of the third layer 112 may work in conjunction with the phase change material of the first layer 108 and the second layer 110 to control the experimental temperature of the experimental material. The insulating material may be a material that is configured to reduce the transfer of thermal energy between the phase change material and the environment (e.g., the air and/or the workbench). In some example implementations, the insulating material may comprise vacuum insulated panels (VIPs), expanded polystyrene (EPS) foam, and polyurethane (PUR) foam. Other insulating material is also contemplated. The third layer of the insulating material may be provided on an outermost surface of the structural holder to substantially reduce the transfer of heat from the phase change material.

In some example implementations, the first layer 108 is disposed on the surface 104 of the structural holder 102, the second layer 110 is disposed on the first layer, and the third layer 112 is disposed on the second layer. However, other arrangements of the layers may also be contemplated. Further, the three layers may also extend up to and be in contact with the raised edge 106, such that the first layer may be disposed on the raised edge adjacent the surface of the structural holder, the second layer may be disposed on the first layer, and the third layer may be disposed on the second layer.

Further, in some other example implementations, the structural holder 102 may comprise a base 120 including the surface 104 and a lid 122 configured to engage the base, the base being structured to receive the experimental material on the surface. The base may be able to receive a wide variety of different experimental receptacles such as a microplate, a flask, a reagent bottle, a mold for centrifuge vials or microtubes, and the like, on the surface. The lid may be configured to engage the base so that the experimental receptacle is contained within. The lid may advantageously reduce heat transfer from the experimental receptacle to the surrounding environment by evaporation, conduction to the surrounding air, and resulting convection and also provide easier storage of the apparatus. In some example implementations, either one or both of the base and the lid includes the first layer of the phase change material, the second layer of the reflective material, and the third layer of the insulating material. As illustrated in FIG. 1A, for example, both the base and the lid include all three layers.

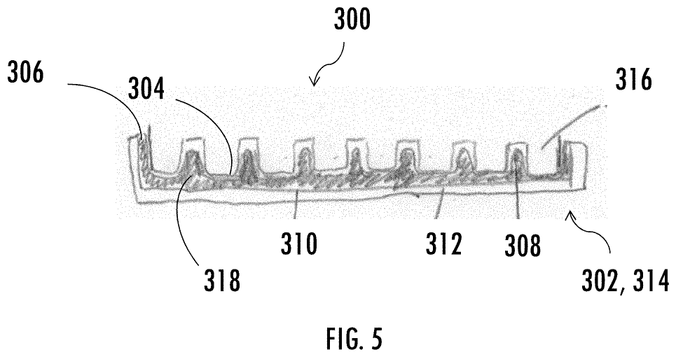

Due to the nature of laboratory conditions it may be desirable to sterilize the structural holder 102, including the layers 108, 110, 112. Therefore, in some example implementations, one or more of the first layer, the second layer, and the third layer may be contained within a sterilizable material. For example, a plastic material such as a plastic bag may be configured to discretely contain each of the three layers. Otherwise, for example, the first layer of the phase change material may be contained in a bottom surface of an experimental receptacle (e.g., a microplate), e.g., FIG. 5. In another example, a paper or polymer wrapping material may be configured to wrap the third layer of the insulating material. In this manner, the sterilizable material may then be able to be sterilized without damaging the layer which it contains.

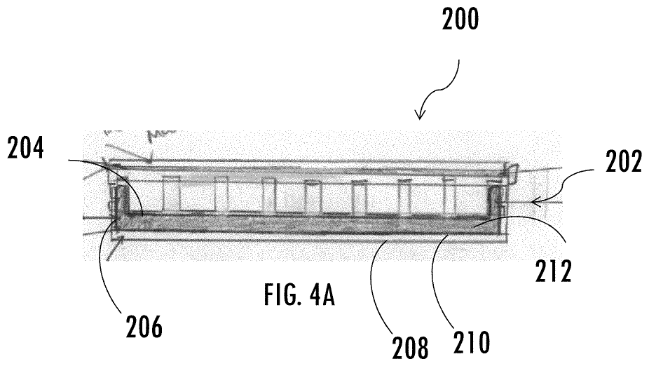

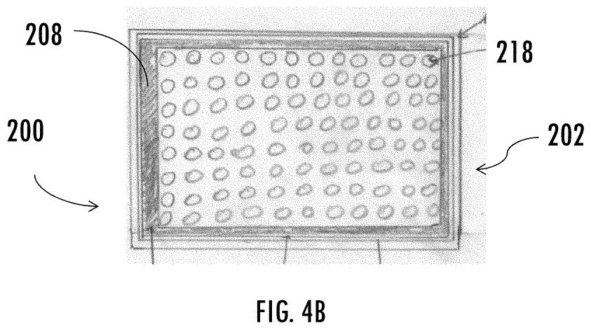

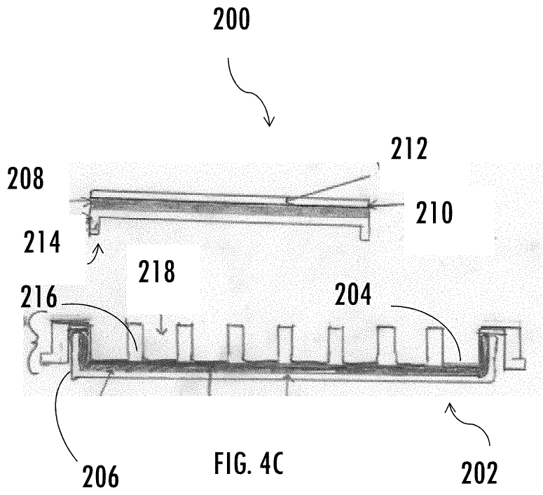

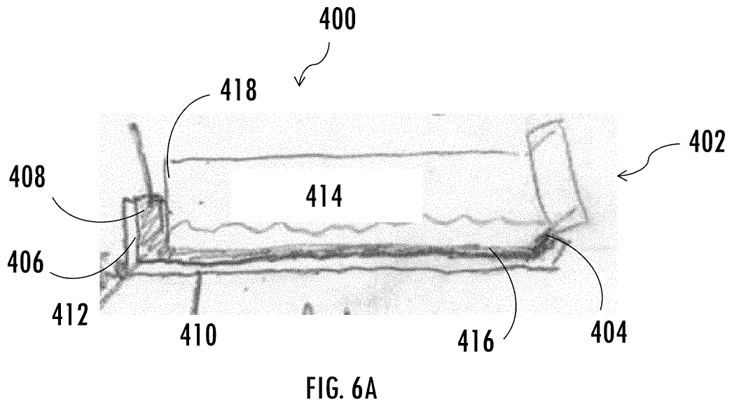

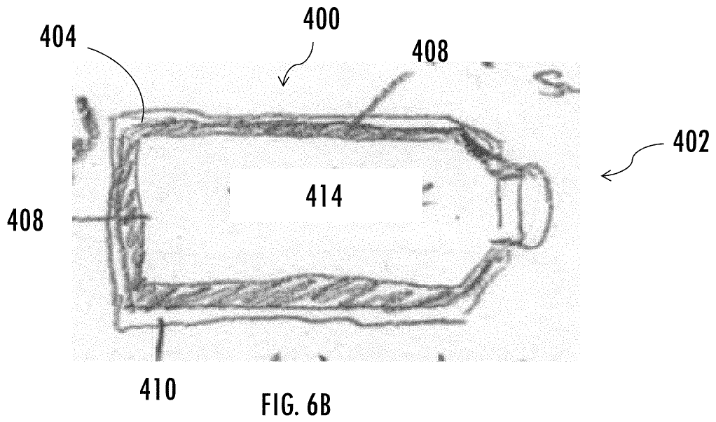

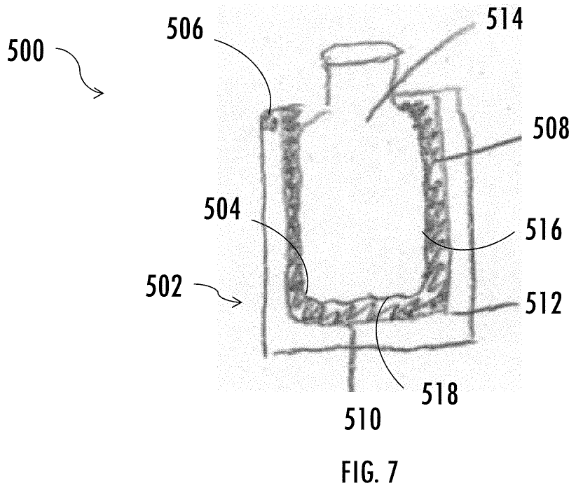

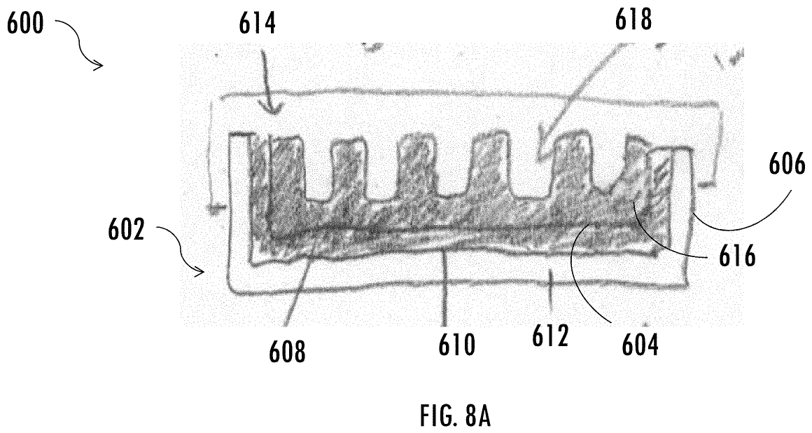

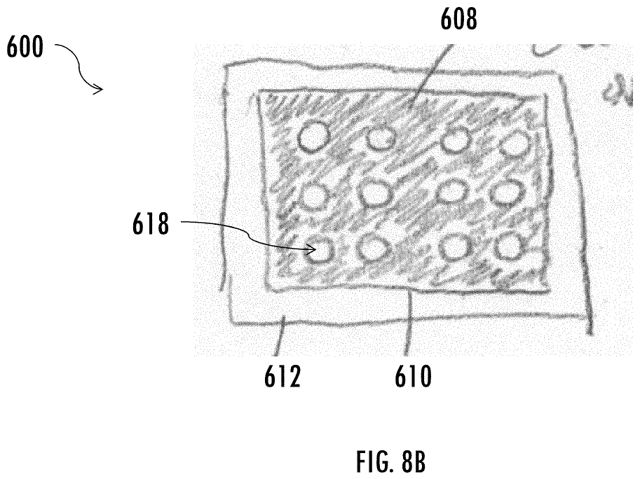

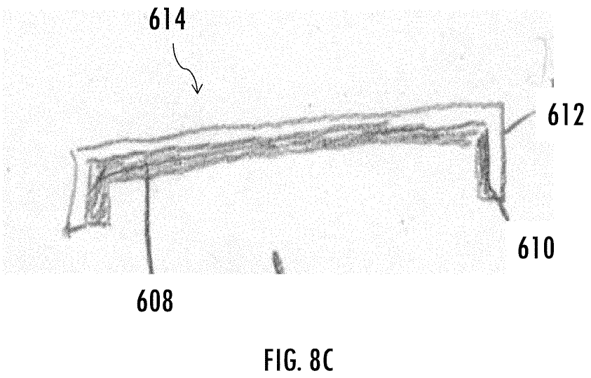

Turning now to FIGS. 4A-8C, example implementations of apparatuses for controlling an experimental temperature of an experimental material are illustrated, where the experimental material is held in different experimental receptacles. FIGS. 4A-4C illustrate an apparatus 200 holding a microplate, FIG. 5 illustrates an apparatus 300 holding a microplate integrated with a structural holder of an apparatus as disclosed herein, FIGS. 6A-6B illustrate an apparatus 400 holding a flask, FIG. 7 illustrates an apparatus 500 holding a bottle, and FIGS. 8A-8C illustrate an apparatus 600 holding a mold for centrifuge vials or microtubes. Other experimental receptacles are also contemplated herein.

In FIGS. 4A-4C, an apparatus 200 for controlling an experimental temperature of an experimental material is illustrated, the apparatus being substantially similar to the apparatus 100 illustrated in FIGS. 1A and 1B. The apparatus may comprise a structural holder 202 structured to hold the experimental material. The structural holder may include a surface 204 and a raised edge 206 extending about the surface thereof. A first layer 208, a second layer 210 and a third layer 212 of respectively a phase change material, a reflective material and an insulating material may be provided in the apparatus, where the first layer may be disposed on the surface and the raised edge of the structural holder, the second layer may be disposed on the first layer, and the third layer may be disposed on the second layer. A lid 214 configured to engage the structural holder may be provided so structural holder that the experimental material is contained in may be provided. The lid may also include the first layer, the second layer, and the third layer. As illustrated in FIG. 4C, the lid includes all three layers.

In FIGS. 4A-4C, a microplate 216 is illustrated, the microplate defining a plurality of wells 218 configured to receive the experimental material therein. The plurality of wells may be provided in a spaced apart configuration on the microplate such that the experimental material may be discretely received in each of the plurality of wells and does not contact any experimental material provided in another well. The microplate may thus be configured to be received on the surface 204 of the structural holder 202, between the raised edge 206, so that the microplate is in contact with the first layer 208 on the surface and the raised edge.

In FIG. 5, an apparatus 300 for controlling an experimental temperature of an experimental material is illustrated, the apparatus being substantially similar to the apparatus 100 illustrated in FIGS. 1A and 1B. The apparatus may comprise a structural holder 302 structured to hold the experimental material. The structural holder may include a surface 304 and a raised edge 306 extending about the surface thereof. A first layer 308, a second layer 310 and a third layer 312 of respectively a phase change material, a reflective material and an insulating material may be provided in the apparatus, where the first layer may be disposed on the surface and the raised edge of the structural holder, the second layer may be disposed on the first layer, and the third layer may be disposed on the second layer. A lid configured to engage the structural holder may be provided so that the experimental material is contained therein. The lid may also include the first layer, the second layer, and the third layer.

In FIG. 5, a microplate 314 is illustrated, the microplate defining a plurality of wells 316 configured to receive the experimental material therein. The plurality of wells may be provided in a spaced apart configuration on the microplate such that the experimental material may be discretely received in each of the plurality of wells and does not contact any experimental material provided in another well. In some example implementations, the microplate may be configured to be integrally formed with the structural holder 302, such that the first layer 308 may be in thermal conductive contact with a bottom surface 318 of the microplate including the plurality of wells. More particularly, for example, the microplate and the structural holder may be the same structure such that the first layer conforms to the microplate. As illustrated in FIG. 5, the first layer conforms to the bottom surface of the microplate, which includes the plurality of wells. This may be advantageous as the first layer of the phase change material substantially conforms to each of the plurality of wells and “wraps” thereabout to improve heat conduction of the apparatus.

In FIGS. 6A and 6B, an apparatus 400 for controlling an experimental temperature of an experimental material is illustrated, the apparatus being substantially similar to the apparatus 100 illustrated in FIGS. 1A and 1B. The apparatus may comprise a structural holder 402 structured to hold the experimental material. The structural holder may include a surface 404 and a raised edge 406 extending about the surface thereof. A first layer 408, a second layer 410 and a third layer 412 of respectively a phase change material, a reflective material and an insulating material may be provided in the apparatus, where the first layer may be disposed on the surface and the raised edge of the structural holder, the second layer may be disposed on the first layer, and the third layer may be disposed on the second layer. A lid configured to engage the structural holder may be provided so that the experimental material is contained therein. The lid may also include the first layer, the second layer, and the third layer.

In FIGS. 6A and 6B, a flask 414 is illustrated, the flask configured to receive the experimental material therein. The flask may comprise a standard cell culture flask, which may be in one of a variety of different sizes, neck designs, cap designs, etc. For example, the flask may comprise a shape with a rectilinear form with a flat bottom surface (bottom surface) and sides surrounding and extending from the flat bottom surface, a curved form with a rounded bottom surface (bottom surface) and sides surrounding and extending from the curved bottom surface, etc. In some example implementations, the flask may be received on the surface 404 of the structural holder 402 within the raised edge 406 thereof and in contact with the first layer 408 on the surface and the raised edge. Depending on the design of the flask, a lateral surface 416 of the flask or a bottom surface 418 of the flask may be received on the surface 404 of the structural holder 402. As illustrated in FIG. 6A, the lateral surface of the flask is received on the surface of the structural holder. As such, whichever surface the flask is received on, the adjacent perpendicular surface may have a height equal or substantially equal the raised edge of the structural holder and/or the shape of the flask.

In FIG. 7, an apparatus 500 for controlling an experimental temperature of an experimental material is illustrated, the apparatus being substantially similar to the apparatus 100 illustrated in FIGS. 1A and 1B. The apparatus may comprise a structural holder 502 structured to hold the experimental material. The structural holder may include a surface 504 and a raised edge 506 extending about the surface thereof. A first layer 508, a second layer 510 and a third layer 512 of respectively a phase change material, a reflective material and an insulating material may be provided in the apparatus, where the first layer may be disposed on the surface and the raised edge of the structural holder, the second layer may be disposed on the first layer, and the third layer may be disposed on the second layer. A lid configured to engage the structural holder may be provided so that the experimental material is contained therein. The lid may also include the first layer, the second layer, and the third layer.

In FIG. 7, a bottle 514 is illustrated, the bottle configured to receive the experimental material therein. The bottle may comprise a standard reagent bottle, a small bottle, a petri dish, a vial, and the like, and may be topped by a cap or stopper and may be in one of a variety of different sizes. For example, the bottle may comprise a circular or cylindrical form, with a flat or curved bottom surface (bottom surface) and sides surrounding and extending from the bottom surface. In some example implementations, the bottle may be received on the surface 504 of the structural holder 502 within the raised edge 506 thereof and in contact with the first layer 508 on the surface and the raised edge. Depending on the design of the bottle, a lateral surface 516 of the bottle or a bottom surface 518 of the bottle may be received on the surface 504 of the structural holder 502. As illustrated in FIG. 7, the bottom surface of the bottle is received on the surface of the structural holder. As such, whichever surface the bottle is received on, the adjacent perpendicular surface may have a height equal or substantially equal the raised edge of the structural holder and/or the shape of the bottle.

In FIGS. 8A-8C, an apparatus 600 for controlling an experimental temperature of an experimental material is illustrated, the apparatus being substantially similar to the apparatus 100 illustrated in FIGS. 1A and 1B. The apparatus may comprise a structural holder 602 structured to hold the experimental material. The structural holder may include a surface 604 and a raised edge 606 extending about the surface thereof. A first layer 608, a second layer 610 and a third layer 612 of respectively a phase change material, a reflective material and an insulating material may be provided in the apparatus, where the first layer may be disposed on the surface and the raised edge of the structural holder, the second layer may be disposed on the first layer, and the third layer may be disposed on the second layer. A lid 614 configured to engage the structural holder may be provided so that the experimental material is contained therein. The lid may also include the first layer, the second layer, and the third layer. As illustrated in FIG. 8C, the lid includes all three layers.

In FIGS. 8A-8C, a mold or fixture for one or more centrifuge vial or microtube 616 is illustrated, the mold being configured to receive the experimental material therein. The mold may comprise a fixture designed to conform to a shape of standard test tubes, microtubes, centrifuge vials, and the like, by defining one or more dips 618 configured to receive standard test tubes, microtubes, centrifuge vials, and the like therein. Accordingly, the mold may be configured to receive on centrifuge vial or microtube in a single dip, or may be configured to receive two, three, four, five, six, etc., centrifuge vials or microtubes in each of the corresponding number of dips. As illustrated in FIGS. 8A-8C, however, multiple centrifuge vials or microtubes are receivable in the corresponding number of dips defined in the mold.

Standard centrifuge vials or microtubes may be 1.5 mL, 2 mL, 15 mL, or 50 mL test tubes, such that the one or more dips are so formed to be able to receive these standard sizes. However, in other examples, other size centrifuge vials or microtubes may be received in the one or more dips, as well. The one or more dips may be formed in the mold such that the experimental material is configured to be received therein and is flush to a surface of the mold. As such, the mold may have a height equal or substantially equal the raised edge 606 of the structural holder 602. Accordingly, the mold may be received on the surface 604 of the structural holder within the raised edge in contact with the first layer 608 on the surface and the raised edge. The lid 614 may be engaged with the structural holder when the experimental material in the mold is received on the surface of the structural holder so that a bottom surface of the lid is flush with a top surface of the raised edge. This is illustrated in FIG. 8A.

Many modifications and other implementations of the disclosure set forth herein will come to mind to one skilled in the art to which these disclosure pertain having the benefit of the teachings presented in the foregoing descriptions and the associated figures. Therefore, it is to be understood that the disclosure are not to be limited to the specific implementations disclosed and that modifications and other implementations are intended to be included within the scope of the appended claims. Moreover, although the foregoing descriptions and the associated figures describe example implementations in the context of certain example combinations of elements and/or functions, it should be appreciated that different combinations of elements and/or functions may be provided by alternative implementations without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. An apparatus for controlling an experimental temperature of experimental material, the apparatus comprising:
a structural holder structured to hold the experimental material, the structural holder including a surface to receive the experimental material; and
a first layer, a second layer and a third layer of respectively a phase change material, a reflective material and an insulating material, the first layer disposed on the surface of the structural holder, the second layer disposed on the first layer, and the third layer disposed on the second layer, at least the first layer of the phase change material being configured to control the experimental temperature of the experimental material when held by the structural holder.

2. The apparatus of claim 1, wherein the phase change material comprises an indicator configured to produce a visible indication when a temperature of the phase change material deviates from a target experimental temperature.

3. The apparatus of claim 2, wherein the indicator comprises a plurality of microcapsule objects dispersed throughout the phase change material, and containing a thermochromatic dye configured to change color as the temperature of the phase change material deviates from the target experimental temperature.

4. The apparatus of claim 3, wherein the microcapsule objects have a density lower than the phase change material and thereby float to a surface of the phase change material as the temperature of the phase change material deviates from the target experimental temperature.

5. The apparatus of claim 1, wherein the structural holder comprises a base including the surface and a lid configured to engage the base, the base being structured to receive the experimental material on the surface.

6. The apparatus of claim 5, wherein either or both the base or the lid includes the first layer, the second layer and the third layer.

7. The apparatus of claim 1, wherein one or more of the first layer, the second layer or the third layer is contained within a sterilizable material.

8. The apparatus of claim 1, wherein the structural holder further includes a raised edge extending about the surface thereof, the first layer being disposed on the surface and the raised edge.

9. The apparatus of claim 8, further comprising a microplate defining a plurality of wells configured to receive the experimental material therein, the microplate being received on the surface of the structural holder, in contact with the first layer on the surface and the raised edge.

10. The apparatus of claim 9, wherein the microplate is integrally formed with the structural holder such that the first layer is in thermal conductive contact with a bottom surface of the microplate including the plurality of wells.

11. The apparatus of claim 8, further comprising a flask configured to receive the experimental material therein, and having a height equal the raised edge of the structural holder, the flask being received on the surface of the structural holder within the raised edge thereof, in contact with the first layer on the surface and the raised edge.

12. The apparatus of claim 8, further comprising a bottle configured to receive the experimental material therein, and having a height equal the raised edge of the structural holder, the bottle being received on the surface of the structural holder within the raised edge thereof, an in contact with the first layer on the surface and the raised edge.

13. The apparatus of claim 8, further comprising a mold for centrifuge vials or microtubes configured to receive the experimental material therein, and having a height equal the raised edge of the structural holder, the mold being received on the surface of the structural holder within the raised edge thereof, in contact with the first layer on the surface and the raised edge.

14. The apparatus of claim 1, wherein the phase change material is selected from the group consisting of at a hydrated salt phase change material, an organic phase change material, and a eutectic phase change material.

15. The apparatus of claim 1, wherein the reflective material is selected from the group consisting of at least metallized polyethylene terephthalate (MPET).

16. The apparatus of claim 1, wherein the insulating material is selected from the group consisting of at least vacuum insulated panels (VIPs), expanded polystyrene (EPS) foam, and polyurethane (PUR) foam.

17. The apparatus of claim 1, wherein the second layer of the reflective material comprises two sheets of reflective material defining an air gap therebetween from at least one of a honeycomb core provided between the two sheets of reflective material and a quantity of pressurized air directed between the two sheets of reflective material.

18. The apparatus of claim 1, wherein the phase change material is configured to control the experimental temperature of the experimental material based on at least a current and a target experimental temperature of the experimental material, an initial temperature of the phase change material, a melting point temperature of the phase change material, a mass of the phase change material relative to a mass of the experimental material, thermal properties of the phase change material, and thermal conduction between the phase change material and the experimental material through the structural holder configured to hold the experimental material.

19. The apparatus of claim 18, wherein the melting point temperature of the phase change material is the target experimental temperature of the experimental material so that the experimental temperature of the experimental material is maintained at the melting point temperature when the experimental material is held by the structural holder.

20. The apparatus of claim 18, wherein the melting point temperature of the phase change material is the target experimental temperature of the experimental material, and the temperature and the melting point temperature of the phase change material are adjustable in order to adjust the experimental temperature of the experimental material when the experimental material is held by the structural holder.
